(19) United States
(12) Patent Application Publication
(10) Pub. No.: US 2018/0245111 A1
(43) Pub. Date: Aug. 30, 2018

(54) Method for Preparing Medium-Long-Chain Triglyceride Using Packed Bed Reactor

(71) Applicant: Jinan University, Guangzhou, Guangdong (CN)

(72) Inventors: Yong Wang, Guangzhou (CN); Manman Liu, Guangzhou (CN); Yinglai Teng, Guangzhou (CN); Aijun Li, Guangzhou (CN); Ning Zhang, Guangzhou (CN)

(21) Appl. No.: 15/755,877

(22) PCT Filed: Mar. 25, 2016
(86) PCT No.: PCT/CN2016/077282

§ 371 Date: Feb. 27, 2018

(30) Foreign Application Priority Data
Aug. 28, 2015 (CN) 201510540665.6

(51) Int. Cl. C12P 7/64; C12N 9/20; B01J 8/02

(52) U.S. Cl. CPC C12P 7/6454; C12Y 301/01003; B01J 8/0292; C12N 9/20

(57) ABSTRACT

Disclosed is a method for preparing a medium-long-chain triglyceride using a packed bed reactor, and belongs to the technical field of deep processing and modification of lipids. In the present invention, a medium-chain fatty acid triglyceride and a lipid as raw materials are subjected to a catalyzed transesterification using an immobilized enzyme having a 1,3 position-specificity being capable of catalyzing a transesterification reaction in the packed bed reactor to prepare a medium-long-chain triglyceride; the transesterification product is then subjected to an ethanol extraction to obtain a highly pure medium-long-chain triglyceride product; and the product remaining after the extraction useful as a raw material is backfilled into the reactor and continues to participate in the next batch of preparation of a medium-long-chain triglyceride.

### TECHNICAL FIELD

The present invention belongs to the technical field of deep processing and modification of lipids, and in particular relates to a method for preparing a medium-long-chain triglyceride using a packed bed reactor.

### BACKGROUND ART

Medium-long-chain triglycerides are lipids having both medium chain fatty acid (C6-C12) and long chain fatty acid (C14-C24) structures, wherein triglycerides having an MLM-type structure are the most typical, with positions Sn-1 and Sn-3 being medium chain fatty acids and position Sn-2 being a long chain fatty acid. Medium- and long-chain fatty acids contained in medium-long-chain triglycerides are in reasonable ratios, and the medium-long-chain triglycerides have the advantages of both medium-chain triglycerides and long-chain triglycerides, are capable of reducing potential hazards of natural triglycerides or hazards brought about thereby due to irrational ingestions, improving the nutritional functions of the natural triglycerides, rapidly supplying energy, providing essential fatty acids and reducing body fat accumulation, and so are new type good functional lipids. In addition, the metabolites thereof can improve the nitrogen balance in vivo, improve the bioavailability of nutrients, reduce the production of inflammatory mediators, maintain the normal phospholipid composition of cell membranes, can be used for fat emulsions under clinical injection, improving defects of natural lipids in drug applications, and are important components of parenteral nutrition.

A traditional method for the synthesis of a medium-long-chain triglyceride is a chemical synthesis. The reaction conditions of this method are severe, during this process numerous by-products are generated, the procedure is tedious and complex, pollution is easily caused to the environment, and the distribution of a certain fatty acid in the glycerol molecule during the reaction is random. Since the localization and distribution of a fatty acid is the key to the metabolism and function of a structural lipid in vivo, the application of the chemical synthesis method has a severe limitation. In contrast, the enzymatic method can directionally change the location distribution of a fatty acid in the glycerol backbone, thus overcome the multiple adverse factors of a chemical catalysis method, and is thus an economical, green and safe production method. With the development of the enzyme preparation industry, the catalytic potencies of lipases are greatly increased, and the enzyme immobilization technology also greatly enhances the reusability of enzymes. On this basis, the use of an appropriate enzyme reactor can reduce mechanical damages to an enzyme carrier, improve their reusability, and further reduce production costs, in a word, a suitable enzyme reactor could be used for facile production of a high value-added medium-long-chain triglyceride.

Different enzyme reactors have different characteristics, and in practical applications, the choice is made based on the application form of the enzyme, the properties of a substrate and a product and the operation requirements, the reaction kinetics and the mass transfer and heat transfer characteristics, the stability, regeneration and replacement of the enzyme, the plasticity and cost of the application of the reactor, etc. A common enzyme reactor currently used in the production is a stirred tank reactor. During the reaction, mechanical stirring produces a greater shearing force, resulting in the fragmentation of an immobilized enzyme carrier, which in turn leads to the shedding of enzyme molecules, thus affecting the enzyme activity, shortening the service life of the enzyme, reducing the yield and increasing the production cost. In contrast, in a packed bed reactor, immobilized enzyme particles are packed in a reaction column to form a stable column bed, and then a substrate solution flows through the reaction column at a certain flow rate to catalyze the reaction by the enzyme. The reaction mode is high in efficiency, easy to operate, simple in structure, and advantageous in the retention of the enzyme activity, it reduces the production cost, and is suitable for a homogeneous reaction of a low viscosity reaction system.

Chinese invention patent application publication number CN 103891920 A discloses a lipid composition containing medium-long-chain triglycerides and a preparation method therefor, wherein the resulting product, i.e., the lipid composition of the invention, has good cooking properties and can reduce fat accumulation in vivo. However, since an inorganic catalyst sodium methoxide is used in this method, making the medium-long-chain triglyceride component in the product fluctuate greatly, wherein the mass proportion of a triglyceride containing one medium-carbon chain fatty acid acyl is 1-90% of that of all triglycerides, and the distribution of fatty acids in the glycerol backbone is uncontrollable. Moreover, after completion of the reaction, means of adding citric acid and washing, etc., are required to remove catalyst, deodorization, etc., causing the production process to be complex. In addition, due to the possibility that the inorganic catalyst and citric acid may remain as residues in the product, the application range of the obtained medium-long-chain triglyceride product is limited and same cannot be used for medical injection preparations.

Chinese invention patent application publication number CN 101979625 A discloses a method for the synthesis of a triglyceride with a medium/long-chain structure through an enzyme-catalyzed transesterification. In the invention, a medium-carbon chain triglyceride and a long-carbon chain triglyceride are used as raw materials, a lipase TLIM-catalyzed transesterification reaction is used, and the optimum process and parameters for the synthesis of a triglyceride with a middle/long-chain structure are determined. However, the reactor used in this method is still a conventional stirred reaction vessel, so only an intermittent reaction can be carried out; moreover, enzyme particles need to be directly added to the reaction system, which is not conducive to maintaining the activity of the enzyme and the recycling of the enzyme, resulting in a longer reaction time (60-180 min). No mention is made of the recycling of enzyme granules and by-products in the method.

### Technical Problem

In order to solve the shortcomings and deficiencies of the prior art, an object of the present invention is to provide a method for preparing a medium-long-chain triglyceride using a packed bed reactor.

### Solution to the Problem

### Technical Solution

The object of the present invention is achieved by the following technical solution:

a method for preparing a medium-long-chain triglyceride using a packed bed reactor, comprising the following steps:

(1) packing a reaction column having a hot jacket with a certain amount of an immobilized enzyme to prepare an enzyme-packed bed reactor, then starting a hot bath pump and a constant temperature hot bath to perform a circulating water bath heating on the packed column, and heating the packed column to a desired reaction temperature;

(2) taking a certain amount of a medium-chain fatty acid triglyceride and a certain amount of a lipid as reactants;

(3) after the packed column is preheated to the desired reaction temperature, starting a constant flow pump connected to the packed column so that the reactants enter the enzyme-packed bed reactor from the bottom of the packed column to initiate an enzyme-catalyzed reaction, and collecting a crude transesterification product flowing from the upper portion of the packed column; and after the reaction is carried out for a certain time, shutting down the constant flow pump and stopping the circulating water bath heating; and

(4) extracting the crude transesterification product obtained in step (3) with ethanol, and performing centrifugation after the extraction to obtain an upper layer alcohol phase and a lower layer oil phase; and taking the lower layer oil phase, and subjecting the lower layer oil phase to a reduced pressure distillation to remove the residual solvent, so that the highly pure medium-long-chain triglyceride is obtained.

The temperature of said reaction in step (1) is 45-80° C., preferably 75° C.; said immobilized enzyme is an immobilized enzyme having a 1,3 position-specificity being capable of catalyzing a transesterification reaction, preferably Novozymes Lipozyme TLIM immobilized lipase.

The number of carbon atoms in fatty acid branches of said medium-chain fatty acid triglyceride in step (2) is 6-12, and caprylic capric glyceride rich in caprylic acid (C8) and capric acid (C10) is preferred. Said lipid is a natural oil rich in long-chain fatty acids (C14-24), preferably soybean oil or sunflower seed oil.

The mass ratio of said medium-chain fatty acid triglyceride to said lipid in step (2) is 1:2 to 2:1, and 45:55 is preferred where caprylic capric glyceride and soybean oil are used.

The flow rate of said reactants in step (3) is in a range of 1.0-30 mL/min, preferably 1.4 mL/min; and said reaction time is 15-60 minutes.

The mass concentration of said ethanol in step (4) is 75-95%; the mass ratio of the crude product to the ethanol is 1:1 g/mL to 1:9 (g/mL), preferably 1:6 to 1:9.

The upper layer alcohol phase obtained after said centrifugation in step (4) is evaporated to remove the residual solvent and can be sent as a reaction raw material back to step (2).

### BENEFICIAL EFFECTS OF THE INVENTION

### Beneficial Effects

Compared with the prior art, the present invention has the following advantages and beneficial effects:

(1) a packed bed reactor loaded with an enzyme catalyst is used in the present invention to catalyze the transesterification reaction. After the completion of the reaction, the immobilized enzyme in the reaction column does not have to be taken out and can be reused multiple times in intermittent production of different batches, or may even be used directly in continuous productions, thereby reducing the reaction cost; and under optimal conditions, the transesterification reaction can still proceed smoothly when the umber of reaction batches reaches 20, and the content (i.e., mass percentage, hereinafter the same) of the medium-long-chain triglyceride can still reach not less than 70%.

(2) The present invention can significantly shorten the reaction time. It originally takes 4 hours for the preparation of a medium-long-chain triglyceride through stirring reaction; the packed bed reactor shortens the reaction time from 15 to 60 minutes, improving the reaction efficiency;

(3) after extraction, the crude product resulting from the enzyme-catalyzed transesterification can be stood or subjected to centrifugation layering, and the supernatant liquid, i.e., a by-product of ethanol extraction, can all be sent as a raw material back to the transesterification reaction in step (2) after the removal of the solvent.

(4) The key of the present invention is to realize the stable preparation of a product containing not less than 75% of a medium-long-chain fatty acid triglyceride through the simple transesterification reaction and the subsequent ethanol extraction. In contrast, a general enzymatic reaction can only give a product of about 68%.

### BRIEF DESCRIPTION OF THE DRAWINGS

FIG. 1 is a schematic diagram of the structural composition of several long-chain triglycerides.

FIG. 2 is a reaction equation for the preparation of a medium-long-chain triglyceride through a transesterification catalyzed by a lipase having a 1,3-specificity in the present invention.

FIG. 3 is a schematic diagram of the packed bed reactor apparatus used in the present invention.

FIG. 4 is a process flow diagram of the highly pure medium-long-chain triglyceride of the present invention.

FIG. 5 is the detection result pattern of the medium-long-chain triglyceride prepared in the present invention through gas chromatography tandem mass spectrometry.

FIG. 6 is the change of the relative activity of the enzyme in the immobilized enzyme reaction column after 20 batches of reaction in an intermittent reaction mode in the present invention.

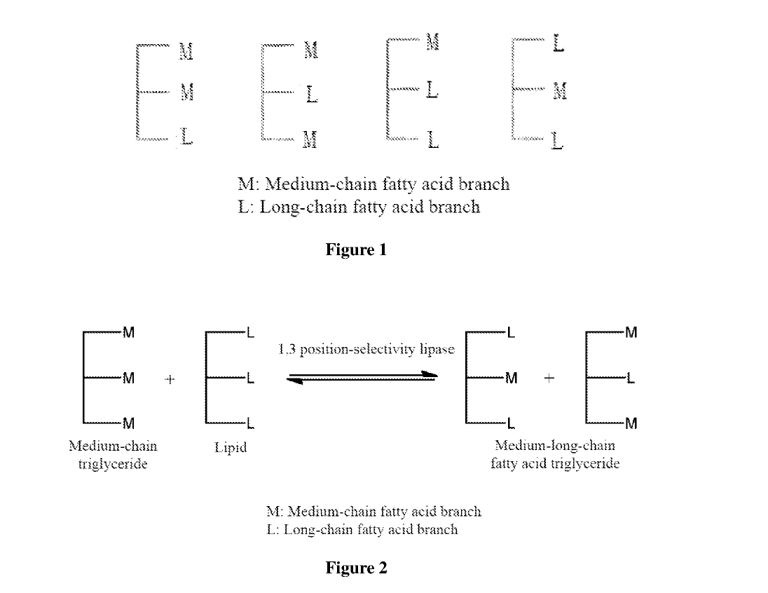

### DETAILED DESCRIPTION OF EMBODIMENTS

The present invention is further described in detail below in conjunction with embodiments and the drawings, but the embodiments of the present invention are not limited thereto.

### Embodiment 1

A method for preparing a medium-long-chain triglyceride using a packed bed reactor, as shown in FIG. 4, comprising the following steps:

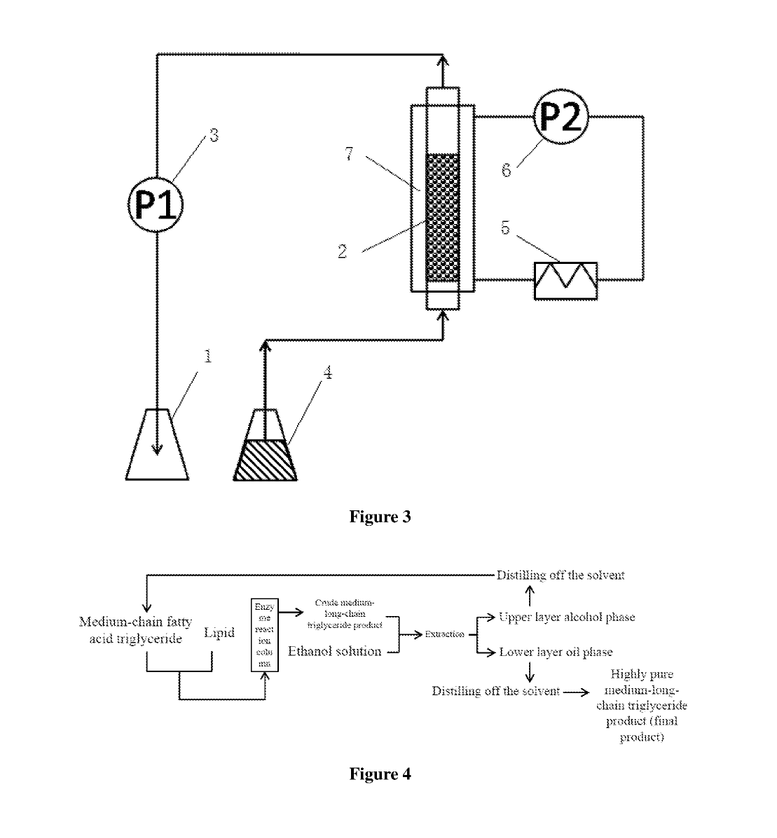

(1) a glass reaction column containing a hot jacket 7 is packed with a certain amount of Novozymes immobilized lipase Lipozyme TL IM to prepare an enzyme-packed bed reactor, the schematic diagram of said enzyme-packed bed reactor apparatus being as shown in FIG. 3, i.e., comprising a product tank 1, a packed column 2, a constant flow pump 3, a substrate tank 4, a constant temperature hot bath 5 and a hot bath pump 6, wherein the product tank 1, the packed column 2, the constant flow pump 3 and the substrate tank 4 are connected in order, and the constant temperature hot bath 5, the hot bath pump 6 and the hot jacket 7 are connected in a circulated manner.

(2) A mixture of 45.0 g of caprylic capric glyceride and 55.0 g of soybean oil is taken as a reaction substrate and added into the substrate tank 4.

(3) The constant temperature hot bath 5 is started and set to a temperature of 75° C.; the hot bath pump 6 connected to the constant temperature hot bath 5 is started for circulating water bath heating to preheat the packed column 2; after the packed column 2 is fully preheated, the constant flow pump 3 is started, the flow rate is set to 1.4 mL/min, so that the liquid reaction mixture in the substrate 4 enters the enzyme-packed bed reactor from the bottom of the packed column 2 to initiate an enzyme-catalyzed reaction, and a crude transesterification product flowing from the upper portion of the packed column 2 is collected by the product tank 1; after the reaction is carried out for 60 min, the constant flow pump 3 is shut down and the circulating water bath heating is stopped; and the resulting crude transesterification product is collected.

The resulting crude transesterification product is detected through gas chromatography tandem mass spectrometry.

The gas chromatography conditions are as follows: DB-1ht capillary column (15 m×0.25 mm, 0.1 μm), with a sample injection port temperature of 380° C., and a split ratio of 40:1, and a pressure of 20 psi; a highly pure N₂ carrier gas, with a flow rate of 4.34 mL/min; a detector temperature of 380° C., a H₂ flow rate of 30 mL/min, and an air flow rate of 300 mL/min; and a sample injection amount of 0.5 μL. Column compartment temperature programming: an initial temperature of 50° C., maintained for 1 min, raised to 100° C. at 50° C./min, raised to 220° C. at 80° C./min, raised to 290° C. at 30° C./min, raised to 330° C. at 50° C./min, maintained for 2 min, and finally raised to 380° C. at 50° C./min and maintained for 3 min.

Preparation of a standard stock solution and an internal standard solution: 800 mg of each of a medium-chain triglyceride (i.e., caprylic capric glyceride, in this embodiment) and a long-chain triglyceride (i.e., soybean, in this embodiment) is weighed and placed in a 10 mL volumetric flask, and diluted with acetone to the mark, for used as a standard stock solution. 200 mg of a molecular distilled monoglyceride is additionally weighed and added to the 10 mL volumetric flask, metered with acetone to the mark, and mixed uniformly as an internal standard solution.

Standard curve creating: each of a standard stock solution of the medium-chain triglyceride and a standard stock solution of the long-chain triglyceride is weighed, each of same is diluted with acetone into 5, 10, 20, 30 and 40 mg/mL solutions, and the solutions are mixed with the internal standard solution in an equivoluminal manner, and with solution concentration as the abscissa and the ratio of the peak area of a corresponding component to the peak area of the internal standard substance as the ordinate, a curve is plotted, and a regression curve is obtained by linear fitting.

Sample detection: 1.60 g of the resulting crude transesterification product is weighed and added into a 10 mL volumetric flask, diluted with acetone to the mark and completely dissolved to prepare a test sample solution. The contents in mass percentage of medium-chain and long-chain triglycerides therein are measured through gas chromatography tandem mass spectrometry. The content in mass percentage of the medium-long-chain triglyceride can be calculated by the following equation using an indirect method:

medium-long-chain triglyceride content=100%−medium-chain triglyceride content−long-chain triglyceride content

As measured by the above-mentioned method, the content of the medium-long-chain triglyceride in the crude product obtained after the transesterification reaction is 73.8% (by mass), and the detection result of the medium-long-chain triglyceride through gas chromatography tandem mass spectrometry is as shown in FIG. 5.

(4) The crude transesterification product obtained in step (3) is extracted with ethanol at a mass concentration of 95%, the solid-liquid ratio of crude transesterification product to ethanol (mass volume ratio) being 1:6; and after centrifugation, the product is divided into an upper layer and a lower layer, and the the lower layer oil phase is taken, subjected to a reduced pressure distillation to remove the residual solvent, so that the final product, i.e., the highly pure medium-long-chain triglyceride, is obtained. The yield of the final product (=the total mass of the final product/the total mass of the reaction substrate prior to the transesterification) is 79.2%, and as measured using the gas chromatography tandem mass spectrometry in step (3), the content of the medium-long-chain triglyceride in the final product is 80.1% (by mass).

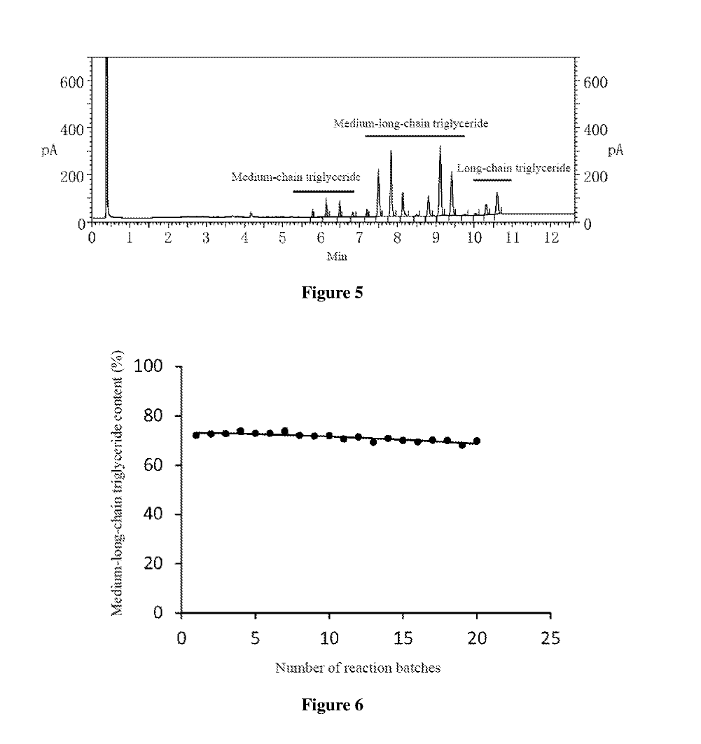

A packed bed reactor loaded with an enzyme catalyst is used in the present invention to catalyze the transesterification reaction. After the completion of the reaction, the immobilized enzyme in the reaction column does not have to be taken out and can be reused multiple times in intermittent production of different batches, or may even be used directly in continuous productions, thereby reducing the reaction cost; The transesterification reaction can still proceed smoothly when the umber of reaction batches according to the above-mentioned steps in this embodiment reaches 20, and the content (in mass percentage) of the medium-long-chain triglyceride in the crude transesterification product can still reach about 70% (as shown in FIG. 6).

### Embodiment 2

This embodiment is the same as Embodiment 1, except the following technical characteristics: the reactant used in step (2) is a mixture of 40.0 g of caprylic capric glyceride and 60.0 g of soybean oil; the reaction temperature in step (3) is 80° C., the flow rate therein is 1.4 mL/min, and the reaction time therein is 60 min; and in step (4), the crude product resulting from the transesterification reaction is extracted with ethanol at a mass concentration of 85%, the solid-liquid ratio of crude product to ethanol being 1:9, and is further subjected to a reduced pressure distillation to obtain the final product.

After detection and calculation with the methods in Embodiment 1, the content of the medium-long-chain triglyceride in the crude product resulting from the transesterification reaction in step (3) in this embodiment is 68.8%; and after extraction and a reduced pressure distillation, the final product is obtained with a yield of 82.1%, the content of the medium-long-chain triglyceride in the final product being 76.8%.

### Embodiment 3

This embodiment is the same as Embodiment 1, except the following technical characteristics: the reactant used in step (2) is a mixture of 45.0 g of caprylic capric glyceride and 55.0 g of soybean oil; the reaction temperature in step (3) is 70° C., the flow rate therein is 1 mL/min, and the reaction time therein is 30 min; and in step (4), the crude product resulting from the transesterification reaction is extracted with a solution of ethanol at a mass concentration of 75%, the solid-liquid ratio of crude product to ethanol solution being 1:9, and is further subjected to a reduced pressure distillation to obtain the final product.

After detection and calculation with the methods in Embodiment 1, the content of the medium-long-chain triglyceride in the crude product resulting from the transesterification reaction in step (3) in this embodiment is 67.0%; and after extraction and a reduced pressure distillation, the final product is obtained with a yield of 80.4%, the content of the medium-long-chain triglyceride in the final product being 75.7%.

### Embodiment 4

This embodiment is the same as Embodiment 1, except the following technical characteristics: the reactant used in step (2) is a mixture of 50.0 g of caprylic capric glyceride and 50.0 g of soybean oil; the reaction temperature in step (3) is 65° C., the flow rate therein is 30 mL/min, and the reaction time therein is 60 min; and in step (4), the crude product resulting from the transesterification reaction is extracted with ethanol at a mass concentration of 85%, the solid-liquid ratio of crude product to ethanol being 1:3, and is further subjected to a reduced pressure distillation to obtain the final product.

After detection and calculation with the methods in Embodiment 1, the content of the medium-long-chain triglyceride in the crude product resulting from the transesterification reaction in step (3) in this embodiment is 65.7%; and after extraction and a reduced pressure distillation, the final product is obtained with a yield of 82.5%, the content of the medium-long-chain triglyceride in the final product being 76.3%.

### Embodiment 5

This embodiment is the same as Embodiment 1, except the following technical characteristics: the reactant used in step (2) is a mixture of 45.0 g of caprylic capric glyceride and 55.0 g of soybean oil; the reaction temperature in step (3) is 75° C., the flow rate therein is 8 mL/min, and the reaction time therein is 15 min; and in step (4), the crude product resulting from the transesterification reaction is extracted with ethanol at a mass concentration of 85%, the solid-liquid ratio of crude product to ethanol being 1:1, and is further subjected to a reduced pressure distillation to obtain the final product.

After detection and calculation with the methods in Embodiment 1, the content of the medium-long-chain triglyceride in the crude product resulting from the transesterification reaction in step (3) in this embodiment is 52.3%; and after extraction and a reduced pressure distillation, the final product is obtained with a yield of 81.6%, the content of the medium-long-chain triglyceride in the final product being 75.4%.

### Embodiment 6

This embodiment is the same as Embodiment 1, except the following technical characteristics: the reactant used in step (2) is a mixture of 45.0 g of caprylic capric glyceride and 55.0 g of sunflower seed oil; and the crude product is subjected to an extraction and a reduced pressure distillation to obtain a final product.

After detection and calculation with the methods in Embodiment 1, the content of the medium-long-chain triglyceride in the crude product resulting from the transesterification reaction in this embodiment is 71.6%; and after extraction and a reduced pressure distillation, the final product is obtained with a yield of 80.2%, the content of the medium-long-chain triglyceride in the final product being 79.4%.

### Embodiment 7

This embodiment is the same as Embodiment 1, except the following technical characteristics: the reactant used in step (2) is a mixture of 50.0 g of caprylic capric glyceride and 50.0 g of sunflower seed oil; the reaction temperature in step (3) is 45° C., the flow rate therein is 10 mL/min, and the reaction time therein is 60 min; and in step (4), the product is extracted with ethanol at a mass concentration of 75%, the solid-liquid ratio of crude product to ethanol being 1:2, and is further subjected to a reduced pressure distillation to obtain the final product.

After detection and calculation with the methods in Embodiment 1, the content of the medium-long-chain triglyceride in the crude product resulting from the transesterification reaction in step (3) in this embodiment is 70.1%; and after extraction and a reduced pressure distillation, the final product is obtained with a yield of 75.5%, the content of the medium-long-chain triglyceride in the final product being 76.8%.

The above-mentioned embodiments are preferred embodiments of the present invention, but the embodiments of the present invention are not limited to the above-mentioned embodiments, and any other changes, modifications, substitutions, combinations and simplifications made without departing from the spirit and principle of the present invention should all be equivalent replacement methods and should all be included in the scope of protection of the present invention.

1. A method for preparing a medium-long-chain triglyceride using a packed bed reactor, the method comprising:
(1) packing a reaction column having a hot jacket with a predetermined amount of an immobilized enzyme to prepare an enzyme-packed bed reactor, then starting a hot bath pump and a constant temperature hot bath to perform a circulating water bath heating on the packed column, and heating the packed column to a desired reaction temperature;
(2) taking a predetermined amount of a medium-chain fatty acid triglyceride and a predetermined amount of a lipid as reactants;
(3) after the temperature of the packed column is preheated to the desired reaction temperature, starting a constant flow pump connected to the packed column so that the reactants enter the enzyme-packed bed reactor from the bottom of the packed column to initiate an enzyme-catalyzed reaction, and collecting a crude transesterification product flowing from the upper portion of the packed column; and after the reaction is carried out for a predetermined time, shutting down the constant flow pump and stopping the circulating water bath heating; and
(4) extracting the crude transesterification product obtained in step (3) with ethanol, and performing centrifugation after the extraction to obtain an upper layer alcohol phase and a lower layer oil phase; and taking the lower layer oil phase, and subjecting the lower layer oil phase to a reduced pressure distillation to remove the residual solvent, so that the medium-long-chain triglyceride is obtained.

2. The method according to claim 1, wherein the temperature of the reaction in step (1) is 45-80° C.; and the immobilized enzyme is an immobilized enzyme having a 1,3 position-specificity capable of catalyzing a transesterification reaction.

3. The method according to claim 2, wherein the reaction temperature in step (1) is 75° C., and the immobilized enzyme is Novozymes Lipozyme TLIM immobilized lipase.

4. The method according to claim 1, wherein the mass ratio of the medium-chain fatty acid triglyceride to the lipid in step (2) is 1:2 to 2:1.

5. The method according to claim 1, wherein the number of carbon atoms in fatty acid branches of the medium-chain fatty acid triglyceride in step (2) is 6-12; and the lipid is a natural oil rich in C14-24 long-chain fatty acids.

6. The method according to claim 5, wherein the medium-chain fatty acid triglyceride in step (2) is caprylic capric glyceride, and the lipid is soybean oil or sunflower seed oil.

7. The method according to claim 6, wherein the lipid is soybean oil, and the mass ratio of the caprylic capric glyceride to the soybean oil is 45:55.

8. The method according to claim 1, wherein the flow rate of the reactants in step (3) ranges from 1.0 mL/min to 30 mL/min; and the reaction time is 15-60 minutes.

9. The method according to claim 1, wherein the mass concentration of the ethanol in step (4) is 75-95%; and the mass ratio of the crude transesterification product to the ethanol is 1:1 g/mL to 1:9 g/mL.

10. The method according to claim 1, wherein the upper layer alcohol phase obtained after the centrifugation in step (4) is evaporated to remove the residual solvent and sent as a reaction raw material back to step (2).

11. The method according to claim 1, wherein the temperature of the reaction in step (1) is 45-80° C.

12. The method according to claim 1, wherein the immobilized enzyme is an immobilized enzyme having a 1,3 position-specificity capable of catalyzing a transesterification reaction.

13. The method according to claim 2, wherein the reaction temperature in step (1) is 75° C., and the immobilized enzyme is a 1,3 lipase originating from Thermomyces lanuginosus.

14. The method according to claim 1, wherein the number of carbon atoms in fatty acid branches of the medium-chain fatty acid triglyceride in step (2) is 6-12.

15. The method according to claim 1, wherein the lipid is a natural oil rich in C₁₄-C₂₄ long-chain fatty acids.

16. The method according to claim 1, wherein the flow rate of the reactants in step (3) ranges from 1.0 mL/min to 30 mL/min.

17. The method according to claim 1, wherein reaction time is 15-60 minutes.

18. The method according to claim 1, wherein the mass concentration of the ethanol in step (4) is 75-95%.

19. The method according to claim 1, wherein the mass ratio of the crude transesterification product to the ethanol is 1:1 g/mL to 1:9 g/mL.

20. The method according to claim 1, wherein the mass ratio of the crude transesterification product to the ethanol is 1:6 g/mL to 1:9 g/mL.
